(19) United States
(12) Patent Application Publication
(10) Pub. No.: US 2019/0359927 A1
(43) Pub. Date: Nov. 28, 2019

(54) System and Method to Generate Progenitor Cells

(71) Applicants: The Charles Stark Draper Laboratory, Inc., Cambridge, MA (US); President and Fellows of Harvard College, Cambridge, MA (US)

(72) Inventors: Brett C. Isenberg, Newton, MA (US); Jonathan R. Coppeta, Windham, NH (US); Ryan A. Dubay, Ludlow, MA (US); David T. Scadden, Weston, MA (US); Azeem Sanjay Sharda, Medford, MA (US)

(21) Appl. No.: 16/419,891

(22) Filed: May 22, 2019

Related U.S. Application Data
(60) Provisional application No. 62/674,977, filed on May 22, 2018.

(51) Int. Cl. C12M 1/12; C12M 3/06; C12M 1/00; C12M 1/06; C12N 5/0789; C12M 1/36; C12M 1/34; A61K 35/14; C12M 3/00; C12M 1/42

(52) U.S. Cl. CPC C12M 25/02; C12M 23/16; C12M 23/50; C12M 27/02; C12N 5/0647; C12M 41/48; C12N 2506/11; C12M 27/16; C12M 25/16; A61K 35/14; C12M 23/42; C12M 35/08; C12M 41/12

(57) ABSTRACT

The present disclosure describes a system, device and method for differentiating cells such as, for example, generating ex vivo common lymphoid progenitors (CLPs) from human hematopoietic stem cells (HSCs). The system and method can be fully automated requiring minimal touch input from a user. Once harvested, the CLPs can be transplanted into a patient for cellular immune therapy.

### RELATED APPLICATIONS

This application claims the benefit under 35 USC 119(e) of U.S. Provisional Application No. 62/674,977, filed on May 22, 2018, which is incorporated herein by reference in its entirety.

### BACKGROUND OF THE INVENTION

Cellular immunotherapy can be used in the treatment of various medical conditions, such as cancer or autoimmune diseases. Immunotherapy can restore and boost immune system function and can increase the patient's natural defenses against, for example, cancer.

The gold standard treatment for patients with a wide range of malignant and non-malignant blood disorders is bone marrow transplant. Currently this treatment requires 12-18+ months for a patient's immune system to become fully functional, during which time the patient is susceptible to a host of serious life-threatening infections and/or graft versus host disease (GvHD).

One existing ex vivo technique for generating progenitor T cells from stem and/or progenitor cells exposes stem and/or progenitor cells to Notch ligand Delta-like-4 and vascular adhesion molecule 1 (VCAM-1) under conditions suitable to generate progenitor T cells.

### SUMMARY OF THE INVENTION

The major disadvantage of current bone marrow transplants is the slow rate at which a patient's immune system reconstitutes. During a period of about 12 to 18 months or longer after receiving a bone marrow transplant, a patient is susceptible to primary and secondary infections that often give rise to complications and can lead to higher mortality rates. GvHD is another major concern.

Part of the problem encountered with current bone marrow transplants relates to the use of mature T cells. This severely restricts the de novo production of diverse T cell populations by the thymus, populations that are required for full diversity and clonal selection critical for functional adaptive immunity.

While an ex-vivo approach for generating T cells from stem and/or progenitor cells exposed to Notch ligand Delta-like-4 and vascular adhesion molecule 1 (VCAM-1) has been reported, this technique is slow, requires considerable pre- and post-cells Delta-like-4 culture time and is touch labor intensive. As with conventional bone marrow transplants, this approach tends to generate mature T cells, thus minimizing the likelihood of producing diverse de novo T cell populations in the recipient's thymus.

Embodiments of the invention address at least some of the deficiencies associated with existing techniques. Thus, the system, device and method described herein aim at generating bone marrow cultures that are significantly enriched in T cell progenitors such as common lymphoid progenitors (CLPs). Thymic competent ex vivo derived immune progenitor cells can be transplanted into a subject towards the goal of reconstituting the T cell and B cell populations more rapidly than possible with standard approaches.

In some embodiments, the invention is directed to a method of treating a subject in need of a bone marrow transplant. The method includes administering a bone marrow culture enriched in CPLs, (using a suitable bone marrow transplant technique, for instance) to the subject. In the recipient's thymus, the CLPs present in the enriched culture, in contrast to transplants of mature or more T cells (or even T-cells that are already more differentiated than CPLs), are further differentiated into mature T cells with a diverse antigen profile, allowing for the rapid reconstitution of the functional adaptive immune system.

Various aspects of the invention relate to a system, device and method for differentiating cells. In one embodiment, the system, device and method are used in the differentiation of hematopoietic stem cells (HSCs) to common lymphoid progenitors (CLPs).

In one aspect, the invention features a system that contains a device for conducting a cell differentiation process, for example, the differentiation of HSCs into PLCs. The system can further include reservoirs for providing cells and various fluids to the device and/or for receiving harvested differentiated cells, waste materials, and so forth. Pumps, valves, switches, manifolds and/or conduits are used to transfer materials into and out of the device. Sensors serve to monitor process conditions. Some implementations of the system also include a source for acoustic radiation. A processor can be used in the partial or complete automation of the system.

In another aspect, the invention features a device that can be or can include a microfluidic or a well-based cassette. In some implementations, the device includes a notch ligand, e.g., Notch ligand Delta-like-4 (DLL4), that promotes the cell differentiation.

Various embodiments of the invention relate to a microfluidic device that includes upper and lower flow channels separated by a membrane. The membrane pores are small enough to prevent HSCs from passing through, while allowing cell culture media or other fluids to pass through. The membrane may or may not be treated with a coating that prevents or minimizes non-specific adhesion of cells to the membrane. A notch ligand (e.g., DLL4) is disposed at a bottom surface of the lower channel. In a system such as the system described herein, the cassette interfaces with custom or commercially available pumps that are used to introduce the cells, perfuse the cells during culture and harvest cells once the differentiation operation is completed. By controlling a set of values on the inlet and outlet of the upper and lower channels, fluid can be routed to and from any of the channel ports.

In one implementation, a microfluidic device, e.g., a cassette, includes a first microfluidic channel. The device can include a dividing wall separating the first microfluidic channel from a second microfluidic channel. At least a portion of the dividing wall can include a membrane having pores smaller than a diameter of an HSC. The second microfluidic channel includes a trapping surface opposite the dividing wall. The trapping surface can include a notch ligand configured to induce differentiation of the HSCs into CLPs.

Other embodiments relate to a well-based device.

In one implementation, the device, e.g., a cassette, includes one or more cell wells. The cassette also has a trapping surface configured to receive HSCs, for example. The trapping surface can include a notch ligand configured to induce differentiation of the HSCs into CLPs. The device can further include a cell distribution system for distributing a population of HSCs onto the trapping surface of the one or more well cells. In some implementations, the distribution system is configured to rotate within each of the one or more cell wells. Other suitable distribution techniques can be employed. For instance, the distribution might rely on a linear motion, for example, to distribute HSCs onto the trapping surface.

In some embodiments, the cell distribution system includes a disk having a first face and a second face. The first face can include an inlet and the second face can include a plurality of outlets configured to enable passage of HSCs. The disk is configured to rotate within or above a cell well of the microfluidic cassette. Alternatively, or in addition, the cell well of the microfluidic cassette can be configured to rotate about the disk.

In further embodiments, the cell distribution system includes at least one impeller configured to rotate within each of the one or more cell wells to generate a first shear force in a fluid in each of the one or more cell wells to distribute the population of HSCs. The cell distribution system can also include at least one impeller configured to rotate within each of the one or more cell wells to wash (dislodge) differentiated cells such as CLPs from the trapping surface of each of the one or more cell wells.

In many of its aspects, the invention relates to a method or process that, in one example, is employed to obtain CLPs from HSCs. The process or method can be thought of as comprising three distinct phases: seeding, differentiation and collection.

In one embodiment, the method includes flowing a first fluid that includes HSCs through a first microfluidic channel and into a second microfluidic channel via a membrane disposed between the first channel and the second channel. The method also includes capturing the HSCs on a first face of the membrane. A second fluid is flown through the second microfluidic channel to wash the HSCs from the first face of the membrane. The method further includes distributing the HSCs on a trapping surface opposite the membrane. The trapping surface can include a notch ligand configured to induce differentiation of the HSCs into CLPs. The method can include perfusing a nutrient from the second fluid into the first microfluidic channel via the membrane. A third fluid is flown through the first microfluidic channel to wash cells consisting of, consisting essentially of or comprising CLPs from the trapping surface.

In some cases, the membrane is part of a dividing wall which separates the first microfluidic channel from the second microfluidic channel. In other cases, the membrane constitutes the entire wall separating these two channels.

The first face of the membrane can form a surface of the first microfluidic channel. A notch ligand can be disposed at an opposite surface of the first channel.

In yet other aspects of the invention a method comprising seeding, perfusion/differentiation, and harvest is conducted in a well-based device.

While a suitable notch ligand that can be used is DLL4, other ligands can be employed in addition or alternatively to DLL4. Of particular interest are ligands that are highly expressed during the HSCs to CLPs differentiation phase and can improve directed differentiation to CLPs, especially when compared to ligands that are present at more advanced stages of differentiation.

The immobilization method may be physical adsorption to the surface, capture by immobilized anti-DLL4 antibody, covalent coupling to the substrate, an adsorbed coating on the substrate, etc.

Further aspects of the invention involve a seeding process by which cells (HSCs, for instance) are induced to collect at the nodes or anti-nodes of a standing acoustic wave established in a microfluidic device. Micro- or nanobeads functionalized with DLL4, for instance, can be introduced into the device, inducing the differentiation of HSCs to CLPs. In some implementations, the beads have magnetic properties, making possible the separation of the cells during harvest by immobilizing the beads with a magnet.

The method, system and device described herein can be used to process bone marrow prior to transplantation such that the rate at which de novo T and B cells are generated in the recipient is increased. This can result in a significantly faster recovery of the recipient's immune system, lowering the risk of severe infection or graft versus host disease (GvHD), both being of major concern following bone marrow transplants.

Treating patients with bone marrow that has been enriched with CLPs takes advantage of the body's natural process of generating a mature, diverse and competent T cell population, an essential feature for the development of a functional immune system. This process does not happen if bone marrow enriched with mature T cells are transplanted.

The method, system and device described herein allow for rapid processing of bone marrow. Embodiments of the invention provide a single, fully integrated and automated system that can generate CLP-enriched samples with minimal user input. In many cases, the invention is practiced by medical professionals, in a hospital setting, and requires minimal touch labor, thus improving yield and minimizing handling mistakes.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a block diagram illustrating an example cell culture system according to the present invention.

FIGS. 2-5 are side cross-sectional views illustrating different configurations of a cassette that can be used in the example system illustrated in FIG. 1.

FIG. 6 is a flow diagram illustrating an example method to differentiate cells using the system illustrated in FIG. 1.

FIGS. 7-9 are side cross-sectional views illustrating schematics of an example cassette at different time points during the method illustrated in FIG. 6.

FIGS. 10A, 10B and 10C show, respectively, T cell, B cell and myeloid cell populations as a function of time for various types of bone marrow cultures.

FIGS. 11A and 11B show the distribution of T Cell Receptor V and J segments in the CDR3 β chain of transplanted mice using Simpson's index for mice receiving bone marrow transplants supplemented with 10% CLPs (FIG. 11B), whereby the mice exhibited greater diversity in their T cell repertoires than mice receiving untreated bone marrow (FIG. 11A).

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present disclosure describes techniques suitable for differentiating cells. Specific aspects relate to cell cultures that contain progenitor cells. A progenitor cell is a cell that has a tendency to differentiate into a specific type of cell. Generally, a progenitor cell tends to be more specific than a stem cell, being closer (or pushed) to differentiate into its “target” cell.

In many implementations, the invention involves the differentiation of human hematopoietic stem cells or HSCs (i.e., cells that give rise to other blood cells) into immune progenitor cells, such as common lymphoid progenitors or CLPs (i.e., cells that are considered to be very early or the earliest lymphoid progenitor cells, giving rise to T-lineage cells, B-lineage cells, natural killer (NK) cells). The progenitor cells can be used for cellular immune therapy, e.g., in bone marrow transplants and other applications.

In one example, HSCs isolated from bone marrow allogeneic are induced to differentiate into common lymphoid progenitors (CLPs) by culturing them on a substrate containing the Notch ligand Delta-like 4 (DLL4). Notch ligands are plasma single-pass transmembrane proteins named Delta-like and Serrate/Jagged, which are glycoproteins with a single transmembrane domain. The extracellular domain (ECD) of both Notch receptors and Notch ligands contains numerous epidermal growth factor (EGF)-like repeats which are post-translationally modified by a variety of glycans.

DLL4 can be immobilized on the substrate using various techniques such as, for example: physical adsorption to the surface, capture by immobilized anti-DLL4 antibody or covalent coupling to the substrate or an adsorbed coating on the substrate.

In addition to DLIA, other compounds (e.g., angiopoietin-1) may also be immobilized to the substrate to modulate the differentiation response of the HSCs. Angiopoietins are proteins with important roles in vascular development and angiogenesis. They bind with similar affinity to an endothelial cell-specific tyrosine-protein kinase receptor. Angiopoietin 1 is encoded by the gene ANGPT 1 and has powerful vascular protective effects, suppressing plasma leakage, inhibiting vascular inflammation and preventing endothelial death.

The bone marrow sample may or may not be preprocessed to remove lineage-positive cells (i.e., mature, differentiate blood cells).

A suitable culture medium can be selected to possess attributes aimed at supporting and/or promoting cell growth and differentiations. A non-limiting example of a medium contains the cytokines interleukin-7 (IL-7), FMS-like tyrosine kinase 3 (FLT3), thrombopoietin (TPO), and stem cell factor (SCF).

In the culture medium, the number of CLPs typically peaks between days 3 to 7 of exposure; harvesting can be conducted during this window. The CLP-enhanced cell population can then be administered to a patient via a bone marrow transplant or further processed to alter the relative numbers of CLPs in the population prior to treatment.

Cell culture, differentiation, harvesting and other processes can be conducted in a system that includes a device, also referred to as a “cassette” or “culture cassette” for conducting the cell differentiation, an incubator, a controller and a microfluidic system composed of one or more reservoirs, one or more pumps, one or more conduits, valves, switches, manifolds, and/or other suitable components.

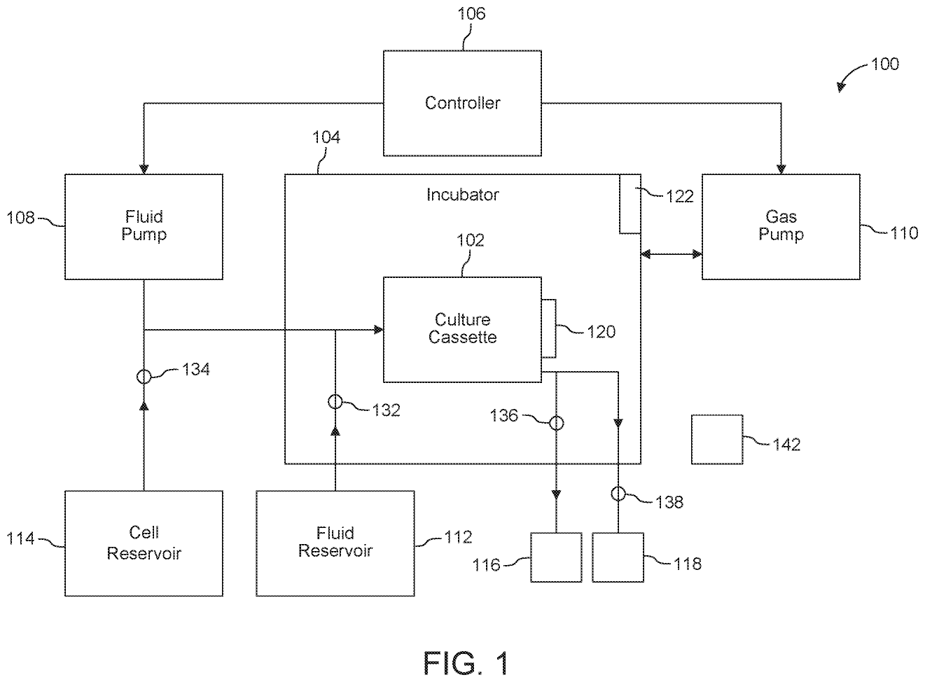

Shown in FIG. 1, for instance, is cell culture system 100 that includes cassette 102, also referred to as culture cassette 102, housed within an incubator 104. The cassette 102 can be a microfluidic device configured to sustain and/or promote the ex vivo differentiation of human hematopoietic stem cells into immune progenitor cells. For example, in the cassette 102, HSCs isolated from bone marrow can be induced to differentiate into CLPs to generate a CLP-enriched sample for implantation into a patient. In many cases, cassette 102 contains a substrate. DLIA and/or another suitable material (e.g., angiopoietin 1) can be immobilized on this substrate. Some cassette designs involve at least two channels: a collection channel and a perfusion channel. In one example, the collection channel is the lower channel and the perfusion channel is the upper channel. Single channel designs are possible in some embodiments that employ an acoustic radiation force to drive cells (and/or other particles) to pressure nodes or pressure antinodes of a standing acoustic wave formed in the fluid channel.

A processor such as controller 106 controls the flow of fluids (culture media, for instance) and gas (e.g., off gas generated in the system) to, through and from the cassette 102.

The fluid flows and gas flows can be driven by at least one fluid pump 108 and at least one gas pump 110, respectively, both of which are under the control of the controller 106. Prior to being directed to, through and out of cassette 102, fluids (cell nutrient media, washing solutions, etc.) can be stored in one or more fluid reservoir(s) 112; cells can be stored in a cell reservoir 114. Waste reservoir 116 can be used to collect spent fluids. Harvested cells (CLPs, for instance) can be collected in reservoir 118.

In more detail, incubator 104 serves to maintain a specific environment within the cassette 102, for example an environment that is suitable for the culture and differentiation of cells and/or tissue. In some implementations, the incubator 104 controls and maintains an environment characterized by one or more parameters such as temperature, humidity, carbon dioxide level, oxygen level, or any combination thereof. For instance, the incubator 104 can be configured or programmed to maintain a standard cell culture environment, as outlined by a cell culture protocol. To illustrate, the incubator 104 can maintain a temperature between about 32° C. and about 37° C. and a humidity between about 50% and about 100%. In one example, the humidity can be maintained at 90% or more to mitigate excessive evaporation. In some implementations, the incubator 104 is configured for the removal of off gases generated by the cells within the cassette 102.

The incubator 104 can include a plurality of access ports. The ports allow sensor connections, flow lines, and/or other lines to pass from the outside environment to the interior of the incubator 104 without affecting the controlled environment within the incubator 104. In some cases, the cell culture system 100 does not include a standalone incubator 104.

Typically, controller 106 is an electronic computing device. For example, the controller can be a laptop, tablet computer, mobile phone or microcontroller. The controller 106 can be a special purposed computer device and can include one or more processors and at least one computer readable medium, such as a hard drive, compact discs, or other storage device. Processor executable instructions are stored on the computer readable medium. When executed, the instructions cause the controller 106 to perform various functions needed to carry out processes described herein.

The controller 106 can include a plurality of inputs and a plurality of outputs through which it interfaces with the various components of the cell culture system 100. The plurality of inputs and outputs of the controller 106 can be digital and/or analog inputs and outputs.

The controller 106 can be configured to control one or more system components and/or conditions (also referred to herein as “parameters”) present in the cell culture system 100. For instance, controller 106 can initiate, terminate or adjust the flow of a fluid into and out of the cassette 102 by controlling the fluid pump 108 and/or valves, switches and the like. Parameters or conditions such as flow rates, pressures, temperatures, gas compositions (e.g., oxygen and carbon dioxide levels), chemical compositions (e.g., drug, toxin and metabolite concentrations), other parameters, and/or combinations of parameters can be controlled using one or more sensors. In some implementations, controller 106 is designed to receive data from a plurality of sensors and to maintain or modify system conditions responsive to the received data.

In FIG. 1, for instance, one or more sensors 120 is/are provided to set, determine, monitor, adjust, optimize, etc. one or more parameters or conditions within cassette 102, while one or more sensors 122 is/are provided to set, determine, monitor, adjust, optimize, etc. one or more parameters or conditions in the interior of the incubator 104. In specific examples, the sensors are used for feedback by the controller 106 in controlling the incubator 104, the fluid pump 108, and the gas pump 110.

The controller 106 can store the sensor and other data on the computer readable medium. In some implementations, the controller 106 can enable a user to set specific system parameters through a user interface. For example, the user can set at which times (e.g., days) fresh media should flow into the cassette 102 from the fluid reservoir(s) 112.

System components can be connected via suitable conduits (e.g., tubing, microchannels, and so forth) that form fluid and/or gas pathways. For instance, conduits 132 and 134 can be used, respectively, to direct materials from reservoirs 112 and 114 to cassette 102; conduit 136 can be used to direct fluids from cassette 102 to waste reservoir 116. Harvested cells can be directed to reservoir 118 through conduit 138. Various switches, valves, flow regulators can be provided to control various flows, e.g., along desired pathways, as further described below.

The reservoirs, pumps, valves, switches, conduits, and similar components can be thought of as forming an arrangement for supplying and/or withdrawing materials to and from device 102. In many embodiments, some or all these components are micro components that are fabricated and/or assembled using microfluidic technology.

In some embodiments, system 100 also includes an acoustic energy source 142 (e.g., a piezoelectric transducer, acoustic wave actuator) for supplying acoustic radiation pressure to a fluid in cassette 102. In some implementations, acoustic radiation forces are applied to drive cells or other particles to nodes or antinodes of a standing wave formed in cassette 102.

System 100 can be operated manually, or in a partially or fully automated mode. The partial and, in particular, the full automation that can be achieved with system 100 reduces or minimizes the touch labor required, thus improving yield and minimizing errors.

Many aspects of the invention relate to the design and operation of cassette 102. In some implementations, cassette 102 is configured to include a plurality (two or more) of microfluidic channels and/or microfluidic wells. The channels and/or wells can include a trapping surface provided with a notch ligand, such as DLL4. For many implementations, the notch ligand is immobilized (also referred to herein as “attached”) onto the trapping surface. Notch ligands can be immobilized by physical adsorption into the trapping surface, captured by an anti-DLL4 antibody, or by covalent coupling to the trapping surface. In addition to the DLL4, other compounds can be immobilized onto the trapping surface. The compounds can be selected to modulate the differentiation response of the HSCs. Examples include but are not limited to angiopoietin 1, Anti-Integrin α9β1 antibody, anti-CD34 antibody and others.

The HSCs can be flowed into or dispensed into the cassette 102. The cassette 102 can include a distribution system that can distribute the HSCs across the trapping surface. The HSCs can bind with the DDL4 and differentiate into CLPs. The CLPs can be extracted from the cassette 102 via a fluid flow or another suitable technique. The CLPs can be administered to a patient, e.g., via a bone marrow transplant.

In many embodiments, the cassette has upper and lower flow channels separated by a membrane. The membrane pores are small enough to prevent HSCs from passing through while allowing cell culture medium to freely pass. The membrane may or may not be treated with a coating that prevents or minimizes non-specific adhesion of cells to the membrane. The bottom surface of the lower channel contains the immobilized DLL4. The cassette interfaces with custom or commercially available pumps that are used to introduce the cells, perfuse the cells during culture and harvest them once differentiated. By controlling a set of values on the inlet and outlet of the upper and lower channels, fluid can be routed to and from any of the port channels.

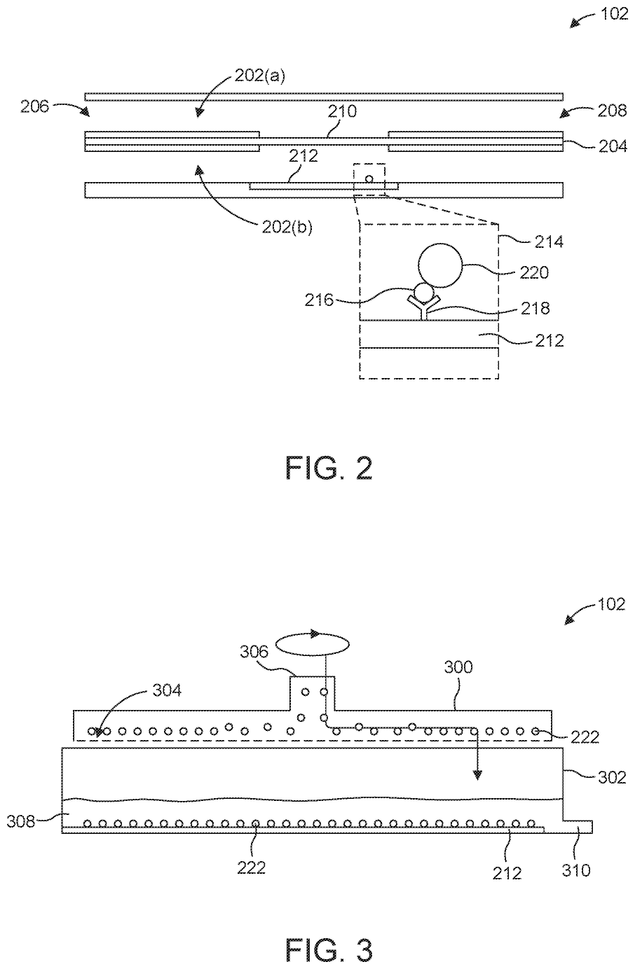

Further aspects of the invention relate to a microfluidic device for conducting cell differentiation processes such as the differentiation of HSCs to PLCs. Several nonlimiting embodiments are illustrated in FIGS. 2-5.

Specifically, FIG. 2 shows a micro-channel cassette 102 that includes microfluidic channels 202(a) and 202(b), collectively referred to as microfluidic channels 202. The microfluidic channels 202(a) and 202(b) can be separated by a dividing wall 204. Each microfluidic channel 202 can include an inlet 206 and an outlet 208. Each of the microfluidic channels 202 (i.e., channels 202(b) and 202(a) in FIG. 2) can be provided with valves or another suitable means for controlling flow to the inlets 206 and from the outlets.

At least a portion of the dividing wall 204 can include a membrane 210 that allows fluid communication between the microfluidic channel 202(a) and the microfluidic channel 202(b). More than two microfluidic channels (separated by a membrane such as membrane 210), can be employed.

At least one wall of one of the microfluidic channels 202 can include a trapping surface 212. As the enlarged view 214 illustrates, the trapping surface 212 can include a notch ligand 216 that is coupled with the trapping surface 212 via an immobilization agent 218. In further embodiments, the immobilization method involves physical adsorption to the surface, covalent coupling to the trapping substrate, an adsorbed coating on the substrate or other suitable techniques.

The microfluidic channel 202 including the trapping surface 212 can be referred to as a collection channel. The microfluidic channel 202 that does not include the trapping surface 212 can be referred to as a perfusion channel. The cassette 102 can include a plurality of collection channels that are defined in a first layer of material and a plurality of perfusion channels that are defined in a second layer of material. In other implementations, the cassette 102 includes a plurality of collection channels in a first layer of material and a single (or a number less than the number of collection channels) perfusion channel that spans the total width of the collection channels formed in a second layer.

The fluid pump 108 can include between about 2 and about 1000 microfluidic channels 202, between about 2 and about 500 microfluidic channels 202, between about 2 and about 250 microfluidic channels 202, between about 2 and about 100 microfluidic channels 202, or between about 50 and about 100 microfluidic channels 202. Suitable channel dimensions can be employed. For example, each microfluidic channel 202 can be between about 1 millimeter (mm) and about 20 mm, between about 1 mm and about 15 mm, between about 1 mm and about 10 mm, between about 3 mm and about 6 mm wide. Each microfluidic channel 202 can be between about 100 micrometer (μm) and about 1000 μm, between about 100 μm and about 800 μm, between about 100 μm and about 600 μm, between about 200 μm and about 400 μm, or between about 200 μm and about 300 μm deep. Each microfluidic channel 202 can be between about 30 mm and about 200 mm, between about 30 mm and about 150 mm, between about 50 mm and about 100 mm, or between about 50 mm and about 75 mm long.

The microfluidic channels 202 can be machined into one or more layers of a hard plastic, glass, or other suitable material. For example, the microfluidic channels 202 can be machined into one or more layers of poly(methylmethacrylate) (PMMA), polystyrene, polysulphone, ultem, cyclo-olefin polymers (COC/COP), polycarbonate and others. The cassette 102 can be manufactured through micro-machining, injection molding, embossing, or other manufacturing techniques. For instance, the collection channels can be embossed into a first layer and the perfusion channels can be embossed into a second layer. One or more walls of the cassette 102 can be transparent or substantially clear. For example, the components of the cassette 102 can be manufactured from substantially clear materials to form view ports. The view ports can provide a user visual access to the cells within the cassette 102.

The microfluidic channels 202 can be separated by a dividing wall 204. In some embodiments, the wall comprises a membrane. As illustrated in FIG. 2, for instance, at least a portion of the dividing wall 204 includes membrane 210. In other embodiments, the dividing wall 204 consists of or consists essentially of the membrane 210. For example, the membrane 210 can be clamped or secured between a first layer that includes the collection channels and a second layer that includes perfusion channels. The membrane 210 can include polydimethylsiloxane (PDMS), polyethersulfone, polycarbonate, polyimide, silicon, cellulose, polymethylmethacrylate (PMMA), polysulfone (PS), polycarbonate (PC), polyester, another suitable material or a combination of materials.

In typical implementations, the membrane 210 is a porous membrane. The diameter of the pores can be less than the diameter of the cells, e.g., HSCs 220 or other target cells, thus blocking passage of the cells through the membrane. In many cases, the pore diameter is less than 5 μm. The membrane 210 can be treated with a coating that prevents or reduces non-specific adhesion of cells to the membrane 210. Materials that can be employed to form the coating include, for example, pluronic, polyethylene imine/polystyrene sulfonate, etc. Multi-layer depositions can be employed to form the coating.

At least one microfluidic channel 202 can include one or more trapping surfaces 212. The trapping surface 212 can be aligned across from the portion of the dividing wall 204 that includes the membrane 210. The length of the trapping surface 212 can be longer than the length of the membrane 210. In some implementations, the trapping surface 212 can be shifted upstream or downstream of the membrane 210.

The trapping surface 212 can include a plurality of notch ligands 216 that are trapped or otherwise coupled to the surface of the trapping surface 212. The ligands functionalize the trapping surface 212. The notch ligands 216 can be DLL4 such that when HSCs are positioned on the trapping surface 212 and interact with the DLL4, the HSCs differentiate into CLPs. The notch ligands 216 can be immobilized on (or otherwise coupled with) the trapping surface 212 via immobilization agents 218. The immobilization agents 218 can be anti-DLL4 antibodies or covalent coupling between the notch ligand 216 and the trapping surface 212. In some implementations, the trapping surface 212 can physically absorb the notch ligands 216 to immobilize the notch ligands 216 on the trapping surface 212.

The trapping surface 212 can be a surface of a wall of the microfluidic channel 202. The trapping surface 212 can be a removable component of a wall of the microfluidic channel 202. For example, the trapping surface 212 can be a removeable (e.g., disposable) insert that is treated to include the notch ligands 216. In one example, the trapping surface 212 can be a polystyrene insert to which a plasma-based surface modification is applied to couple the notch ligands 216 to the insert. In some implementations, the trapping surface 212 can be treated or coated with other compounds to modulate or control the differentiation of the HSC 220. For example, anti-angiopoietin-1 antibody may also be immobilized to the trapping surface 212. Anti-Integrin α9β1 antibody, anti-CD34 antibody and others also can be used. Many embodiments rely on physical adsorption to tissue culture plastic (e.g., plasma-treated polystyrene). Plasma treatment has been shown to be effective in enhancing adsorption to other plastics as well.

In some cases, in addition to or as a replacement of the membrane 210, the system 100 can employ acoustic trapping to separate and collect cells. This technique can be used in the active selection and manipulation of cells from a static or a dynamic flow within a microfluidic device. In some cases, (e.g., if the membrane 210 is absent) device 102 can be configured to include a single channel.

Typically, standard microfluidic channels accommodate half the acoustic wavelength in the fluid, but quarter wavelength designs can be utilized for cell manipulation within a microfluidic device as well. A standing wave is established within the fluid channel which then exposes cells or other particles to an acoustic radiation force (ARF). This force pushes cells or particles of positive acoustic contrast towards pressure nodes; cells or particles of negative contrast, on the other hand, migrate towards pressure antinodes. Cells of larger size and greater density are driven towards the pressure nodes more readily compared to cells of lower volume and lesser density. Given this information, cell position can be manipulated by tuning frequency, acoustic power, and carrier fluid properties.

The ARF is proportional to the diameter cubed (i.e. volume), acoustic energy density (square of pressure amplitude), and the contrast factor. Minor differences in cell or particle size are amplified by the cubic relationship to the ARF, making this an effective method of moving cells of specific sizes. Acoustic energy density is controlled by pressure waves generated within the fluid, (which are controlled by transducer activation and displacement). The contrast factor incorporates the density and compressibility of the cell or particle relative to the density and compressibility of the carrier fluid. Altering fluid suspending densities allows for additional selectivity of cells or particles within a microfluidic device.

With reference to cassette 102, a standing acoustic wave can be applied using a suitable source (element 142 in FIG. 1). The standing acoustic wave can generate pressure nodes and pressure anti-nodes within the fluid contained in the cassette 102. The cells, e.g., HSCs 220 (as well as other particles) within the fluid are driven to one of the pressure nodes or pressure anti-nodes by an acoustic radiation force generated by the standing waves. As discussed above, cells and particles with a positive contrast factor are driven towards the pressure nodes, while cells and particles with a negative contrast factor are driven toward the pressure antinodes. A cell or particle's contrast factor (and magnitude thereof) can be based on the bulk modulus and the density of the cell or particle. The magnitude of the acoustic radiation force can also be based on the volume of the cell or particle. The rate at which cells and particles move to the pressure nodes or pressure anti-nodes can be based on the magnitude of the acoustic radiation force, which is linearly dependent on the contrast factor and volume of the cell or particle.

Once the cells 222 (e.g., HSCs, for instance) have been collected at the nodes or anti-nodes of the standing acoustic wave, micro- or nanobeads functionalized with DLL4 can be introduced into the cassette 102. Trapped on the (trapping) surface of the functionalized beads, the HSCs are induced to differentiate into, for example, CLPs. In some implementations, the beads can include magnetic properties that can enable separation of the cells during harvest by immobilizing the beads with a sufficiently strong magnet.

The device 102 also can be a well-based device (cassette) that includes one or more wells. A trapping surface containing a notch ligand such as DLL4 can be disposed at a cell wall, for example. In some implementations, the cassette is provided with a distribution system configured to dispense cells and/or fluids into the well. FIGS. 3 through 5 illustrate exemplary embodiments.

Shown in FIG. 3 is well-based cassette 102 provided with distribution system 300. The distribution system 300 can be configured as a sprayer or sprinkler.

The cassette 102 can include the distribution system 300 and a well 302. The cassette 102 can include a plurality of distribution systems 300 and a plurality of wells 302. For example, the wells 302 can be the wells of a multi-well plate. The well 302 can be or include a culture dish. Each well 302 can be associated with a different distribution system 300. In some implementations, the system 100 includes one distribution system 300 that can be robotically moved to and activated over or in each of the wells 302. The wells 302 can have a diameter between about 5 mm and about 75 mm, between about 10 mm and about 50 mm, or between about 15 mm and about 25 mm. The wells 302 can have a depth between about 5 mm about 50 mm, between about 10 mm and about 40 mm, or between about 15 mm and about 30 mm.

The distribution system 300 can include a first surface that includes a plurality of outlets (orifices, for example) 304. The first surface of the distribution system 300 can be a bottom surface of the distribution system 300 that faces toward the floor of the well 302 when the distribution system 300 is positioned above or in the well 302. The outlets 304 can be distributed across the first (e.g., bottom) surface of the distribution system 300. Each of the outlets 304 can have a diameter that enables cells 222 (e.g., HSCs and/or other cells) to pass through the outlets 304. A second surface of the distribution system 300 can include an inlet 306. The second surface can be opposite the first surface. The fluid pump 108 (see FIG. 1) can pump fluid and the cells 222 into the distribution system 300 via the inlet 306.

As illustrated in FIG. 3, cells 222 (e.g., HSCs 222 in FIG. 2) and/or a fluid can enter the distribution system 300 at the inlet 306, distribute throughout the interior of the distribution system 300, and then exit the distribution system 300 through one of the plurality of outlets (orifices or perforations) 304. The volume defined between the first and second surface of the distribution system 300 can be disk shaped. The first surface can have a shape substantially similar to the shape of the floor of the well 302 or the trapping surface 212. For example, the floor of the well 302 can be circular and the trapping surface 212 can also be circular—covering the majority of the well's floor. The first surface can also be circular. The diameter of the distribution system 300 (or the first surface) can be slightly less than the diameter of the well 302 such that the distribution system 300 can spin within the well 302. In some implementations, the distribution system 300 can be bar shaped. Other suitable shapes can be employed.

In some implementations, the distribution system 300 can spin as the cells 222 (e.g., HSCs 220 in FIG. 2) flow into and through the distribution system 300. The distribution system 300 can distribute the cells 222 across the surface of the trapping surface 212. The distribution system 300 can spin above or within the well 302. In some implementations, the distribution system 300 remains stationary as the well 302 rotates around the distribution system 300.

The distribution system 300 can also be used to flow fluid 308 (e.g., a suitable culture medium) into the well 302. The fluid 308 (with or without) the cells 222 can be flowed into the distribution system 300 and through the outlets 304 and into the well 302. The fluid pump 108 can remove waste or old medium through the outlet 310. The fluid pump 108 can circulate new fluid 308 into the well 302 by flowing fresh fluid 308 into the well 302 via the distribution system 300, which dispenses the fluid 308 into the well 302 via the outlets 304.

Fluid flow can also be used to dislodge cells 222 from the trapping surface 212. For example, once the HSCs 220 have differentiated into CLPs, the fluid pump 108 can flow fluid through the distribution system 300 and out the outlets 304 at a rate that dislodges the CLP-enriched cell population from the trapping surface 212. The well 302 can be tilted as the dislodging flow is applied to wash the cells 222 and fluid out of the outlet 310.

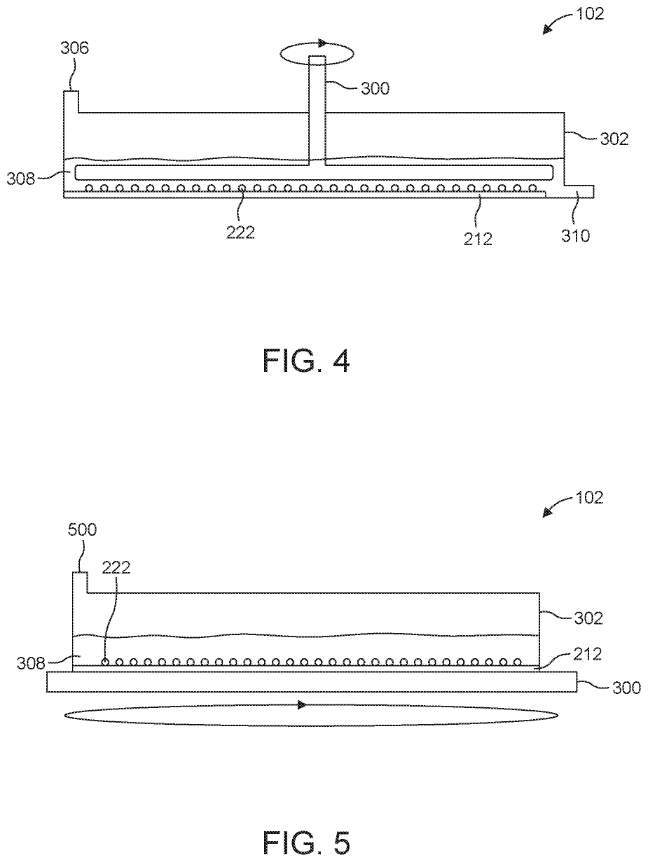

FIG. 4 illustrates a well-based cassette 102 with another example of the distribution system 300. As shown in FIG. 4, the distribution system 300 can be configured as an impeller. The well 302 can include an inlet 306 and an outlet 310. During operation, fluid 308 and cells 222 are introduced into the well 302 via the inlet 306. The distribution system 300, configured as an impeller, can be lowered into the fluid 308 and rotated. The rotation of the impeller can generate a shear force in the fluid 308 that causes the cells 222 to distribute across the trapping surface 212. In some implementations, the well 302 can rotate around a static distribution system 300.

The distribution system 300 also can be used to dislodge the cells 222 from the trapping surface 212. For example, the distribution system 300, as an impeller, can be spun to generate a shear force in the fluid. In many cases, the shear force generated by the distribution system 300 to distribute the cells 222 and/or dislodge the cells 222 from the trapping surface 212 is selected to avoid damaging cells 222. For instance, the shear force used can be below 5 Pa (Pascal) or below about 1 Pa. Once the cells 222 are dislodged from the trapping surface 212 as, for example, CLPs, the well 302 can be tilted to enable the CLPs and fluid to exit the well 302 via the outlet 310.

FIG. 5 illustrates a well-based cassette 102 with still another example of the distribution system 300. Here, the distribution system 300 is configured as a centrifuge, a rotating surface or another type of platform or device that can rotate. In more detail, well 302 is secured to the distribution system 300 and includes a port 500, serving as both an inlet and an outlet to the well 302. During operation, fluid 308 and cells 222 are introduced to the well 302 through port 500. The distribution system 300 rotates and spins the well 302 to distribute cells 222 across the trapping surface 212. Once the cells 222 differentiate (into, for example CLPs) the distribution system 300 can be rotated, spinning the well 302 to dislodge the cells 222 from the trapping surface 212. The cells 222 can collect against the walls of the well 302, where the cells 222 and fluid 308 can be collected via the port 500.

The invention also relates to a method that can be used to differentiate cells, e.g., HSCs into PLCs. In one embodiment, HSCs isolated from bone marrow are induced to differentiate into CLPs by culturing them on a substrate that includes immobilized DLL4. The immobilization method may be physical adsorption to the surface, capture by immobilized anti-DLL4 antibody or covalent coupling to the substrate or an adsorbed coating on the substrate. In addition to DLL4, other compounds (e.g., angiopoietin-1) may also be immobilized to the substrate to modulate the differentiation response of the HSCs. The culture medium can contain cytokines such as interleukin-7 (IL-7), FMS-like tyrosine kinase 3 (FLT3), thrombopoietin (TPO), and stem cell factor (SCF) to support cell growth and facilitate differentiation. The bone marrow sample may or may not be preprocessed to remove lineage-positive cells (i.e., mature, differentiate blood cells). The number of CLPs peaks between days 3-7 in culture and are typically harvested during this window period.

The CLP-enhanced cell population can then be administered to a patient via a bone marrow transplant or further processed to alter the relative numbers of CLPs in the population prior to treatment.

In the context of a microfluidic device that includes micro channels, such as, for instance, cassette 102 (FIG. 2), the seeding phase involves introducing a bone marrow suspension into the device via a lower channel while fluid exits the cassette via the upper channel, resulting in cells that are concentrated in the lower channel. Once all the cells have been pumped into the cassette, this flow is terminated. The cells are induced or allowed to settle to the bottom of the lower channel where they are trapped on a trapping surface.

In the perfusion/differentiation phase, a fluid is introduced at one end of the upper channel and exits at the other end of the upper channel. This flow ensures that the cells are provided with sufficient oxygen and nutrients during the multi-day differentiation process. Because the cells are separated from this flow by a permeable membrane, they receive the nutrient without being directly exposed to flow. This is crucial in situations in which the cells do not adhere to the bottom of the channel and would be washed away if exposed to even small levels of direct flow.

After sufficient time for differentiation has elapsed, flow in the upper channel is stopped and, in the collection phase, the CLP-enriched cell population is collected by applying a shearing flow in the bottom channel that sweeps the non-adherent cells off of the surface and out of the cassette for collection and/or further processing. Typically, the flow rate has a value selected to reduce, minimize or prevent shear-related cell damage. In one example, the maximum wall shear stress is selected to be less than or equal to 1 Pa (Pascal).

This process can be fully-automated such that minimal input/manipulation is required by the user. Integrated cell handling can be used to minimize touch labor and user error and improve consistency.

In some embodiments, the method employs a system such as system 100 and/or a device such as cassette 102.

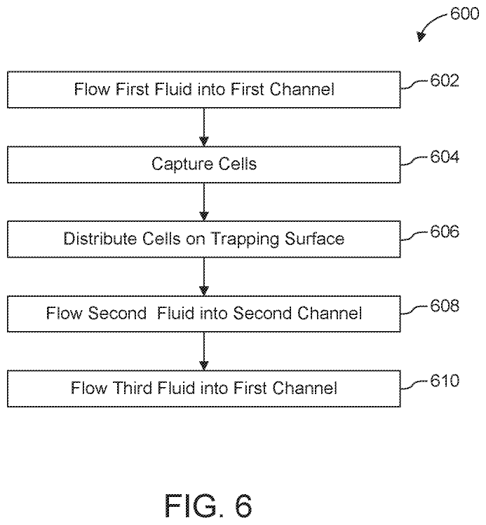

FIG. 6 shows an illustrative method 600 for differentiating cells. Method 600 can include one, more or all of the steps represented by blocks 602, 604, 606, 608, and/or 610. In one embodiment, method 600 includes flowing a first fluid through a first microfluidic channel of a cassette (BLOCK 602); capturing a plurality of cells (BLOCK 604); distributing the cells on a trapping surface (BLOCK 606); flowing a second fluid through a second microfluidic channel of the cassette (BLOCK 608); and flowing a third fluid into the first channel of the cassette (BLOCK 610). The operations identified in BLOCK 606 and 608 can be conducted in the sequence shown in FIG. 6, simultaneously, or by flowing the second fluid through the second microfluidic channel of the cassette (or in other embodiments, another fluid, such as a washing solution), before the cells become trapped onto the trapping surface. In this last approach, the second fluid and/or a washing solution can be used to push cells away from the membrane onto the trapping surface.

The cells can distribute to the trapping surface under the force of gravity once the first flow is stopped. In many cases, the second flow is only initiated once the cells have been collected on the trapping surface.

A third fluid is flown through the first microfluidic channel to release the captured cells.

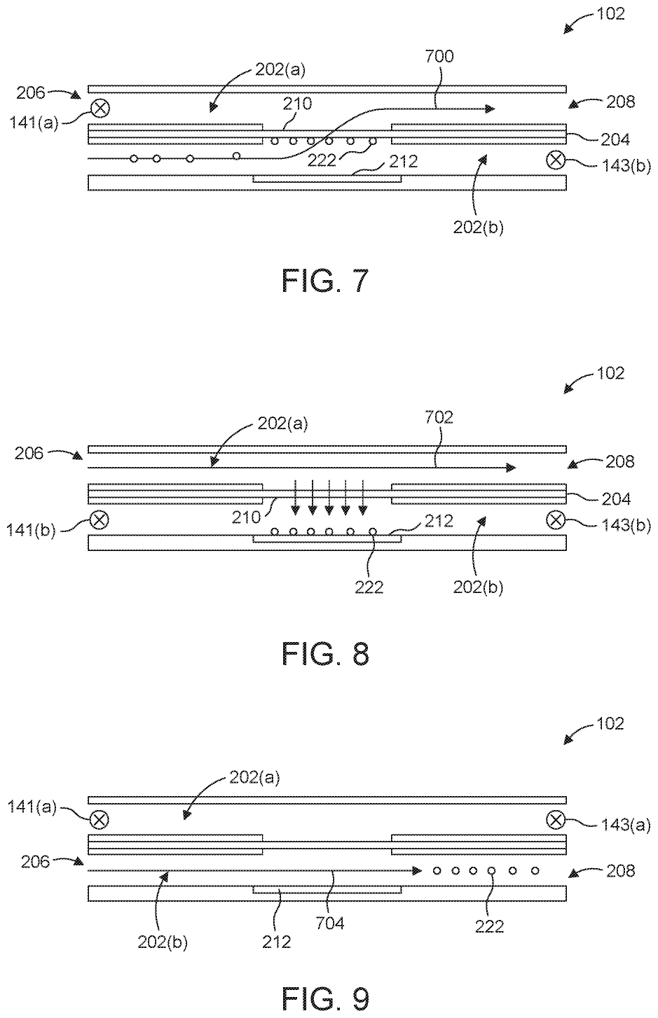

Specific embodiments of method 600 are illustrated in FIGS. 7 through 9. As seen in FIG. 7, the method 600 can include flowing a fluid into a first channel of a microfluidic device (BLOCK 602), such as, for instance, a device configured as a plurality of microfluidic channels. With reference to FIG. 2, the first fluid is introduced into microfluidic channel 202(b)). The first fluid can include a bone marrow, mobilized peripheral blood, cord blood, etc. suspension that includes a population of cells 222, e.g., HSCs 220. The first fluid can also include a buffer or growth medium. Examples include but are not limited to PBS (buffer), DMEM (growth medium).

As seen in BLOCK 604 (FIG. 6), the method 600 illustrated in FIG. 7 also includes capturing or collecting a plurality of cells at or along a first face of a membrane 210, which, as described above, can be a component of dividing wall 204 which separates two microfluidic channels, namely 202(a) and 202(b).

With inlet 206 of microchannel 202(a) and outlet 208 of microchannel 202(b) closed, e.g., by activating, respectively, valves 141(a) and 143(b), valves that can be part of the valve system described with reference to FIG. 1, the flow of the first fluid through and out of the culture cassette 102 follows the path 700. In more detail, the first fluid flows from the inlet 206 of the microfluidic channel 202(b) passes through the membrane 210 and into the microfluidic channel 202(a) and exits the cassette at outlet 208 of the microfluidic channel 202(a). Since membrane 210 has pores smaller than the diameter of the cells 222, cells 222 in the first fluid are collected or captured at or along a surface of the membrane 210 that faces the microfluidic channel 202(b).

Embodiments of the phases identified in FIG. 6 as BLOCKS 606 and 608 are illustrated in FIG. 8 which is a schematic of the micro-channel cassette 102 showing cells trapped on the trapping surface 212 and a second fluid flowing into a second channel (e.g., microfluidic channel 202(a)). In many cases, the cells are detached from the face of membrane 210 and fall onto the trapping surface 212 under the force of gravity. Alternatively, or in addition, the cells 222 can be pushed from the first face of the membrane 210 toward the trapping surface 212, in the direction of the arrows, by the second fluid (or another fluid, e.g., a washing fluid, employed for this particular purpose).

As the cells are detached from the membrane (by gravity forces, for instance) the cells can be captured by or distributed on the trapping surface. The trapping surface can include notch ligands that are configured to bind to or interact with the cells. The notch ligands can be configured to induce differentiation in the captured cells. For example, the captured cells can be HSCs and notch ligands such as DLIA can cause the HSCs to differentiate into CLPs.

In flow mode, both input 206 and output 208 of the first microfluidic channel 202(b) are closed (e.g., by closing valves 141(b) and 143(b) and the second fluid passes through the second microfluidic channel 202(a) along pathway 702.

The second fluid can be a growth medium. The compounds in the second fluid can pass (also referred to herein as “perfuse”) through the membrane 210 and into the microfluidic channel 202(b) where they can interact with the cells 222. Waste from the cells 222 within the microfluidic channel 202(b) can perfuse through the membrane 210 and into the microfluidic channel 202(a) where the second fluid can transport the waste out of the cassette 102.

The cells captured on trapping surface 210 can remain in the cassette for a suitable time period, e.g., between about 3 and about 7 days, as the cells begin to differentiate. During the differentiation process, the cells can be perfused with fresh media from an adjacent microfluidic channel as described above. The perfusion of media from the adjacent microfluidic channel into the microfluidic channel with the trapping surface 212 can be configured to cause substantially no flow in the microfluidic channel that contains the trapping surface 212 (channel 202(b) in FIG. 8.

As differentiation begins and progresses cells 222 will contain HSCs as well as CLPs. In one example, a HSC population is positioned on a surface with immobilized DLL4 and allowed to culture for multiple days (3-7 being optimal in many cases) during which time a subset of the initial population will differentiate into CLPs. During that time, medium needs to be exchanged periodically. In one example, half medium volume is replaced every other day. Other rates for exchanging the medium can be utilized.

In cases in which the second fluid or a washing solution is employed to promote the distribution of cells collected on the membrane onto the trapping surface, the fluid can be introduced at end 206 of channel 202(a), pass through the membrane and exit the device at end 208 of channel 202(b). The operation can be conducted with valves 141(b) and 143(a) being closed. Flow rates and/or volumes can be adjusted to prevent or minimize washing away HSCs before they are trapped at the trapping surface. In other approaches, the second fluid or a washing solution is directed to and through device 102 along pathway 702 (FIG. 8) at a rate and/or volume selected to promote pushing cells away from membrane 210 and onto the trapping surface 212.

The method 600 can include flowing a third fluid through the first microfluidic channel (BLOCK 610) to release the differentiated cells from the trapping surface 212. In the embodiment shown in FIG. 9, valves 141(a) and 143(a), valves that can be components of the valve system described with reference to FIG. 1, are closed and the third fluid flows through the microfluidic channel 202(b) along the path 704.

The fluid flowing through the microfluidic channel 202(b) can generate a shear force on the cells 222 (which now consist of, consist essentially of or comprise CLPs). The shear force can wash the cells 222 from the trapping surface 212 and direct them downstream, toward the outlet 208 of the microfluidic channel 202(b). The shear force can be less than about 10 Pa, less than about 5 Pa, or less than about 1 Pa. The CLPs (or CLP-enriched cell population) can be harvested from the cassette 102 after between about 3 and about 7 days. The CLPs can be harvested from the cassette 102 prior to the differentiation of the HSCs into T cells.

Upon collection from the device the cells may or may not need further concentration before being given to a patient.

In an illustration, the HSCs 220 harvested from cassette 102 are administered to a patient for cellular immune therapy. Prior to transplant, the HSCs 220 can be further processed. For example, the HSCs 220 can be processed to alter the concentration of the HSCs 220 in the cell population that is transplanted into the patient.

Transplanting CLPs into the patient can take advantage of the body's natural process of generating a mature, diverse, and competent T cell populations. Transplanting CLPs can provide better outcomes for patients when compared to conventional bone marrow transplants or implanting bone marrow enriched with mature T cells. The better outcomes can include decreasing recovery time of the recipient's immune system and lowering the risk of severe infection or graft versus host disease (GvHD).

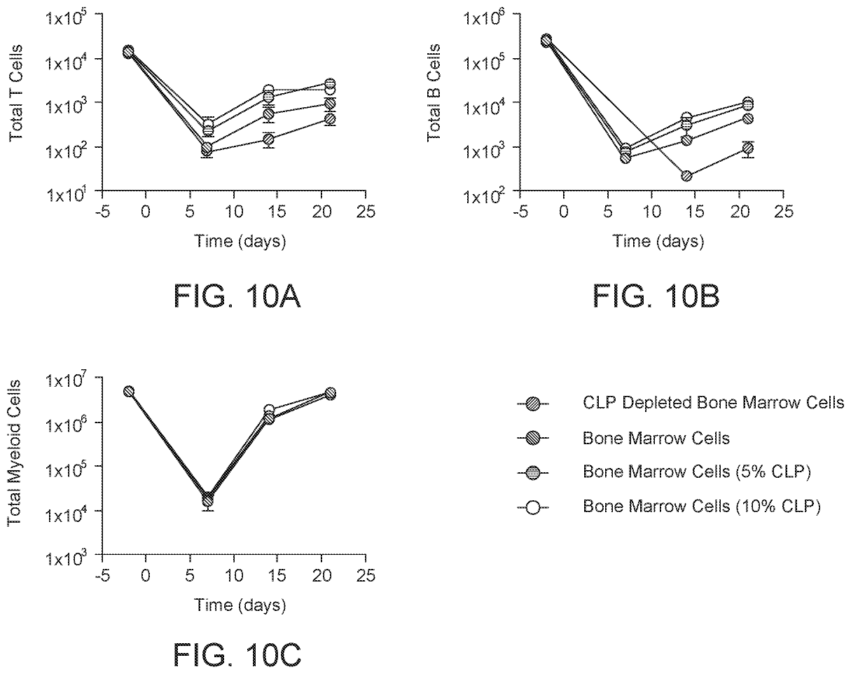

Practicing aspects of the invention are illustrated in FIGS. 10A, 10B and 10C, showing, respectively, the total numbers of T cells, B cells and myeloid cells that are produced, as a function of time from bone marrow cells, bone marrow cells depleted in CPLs, bone marrow cells enriched with 5% CPLs and bone marrow cells enriched with 10% CPLs. These figures demonstrate the ability to more rapidly reconstitute the T and B cell populations relative to current bone marrow methods.

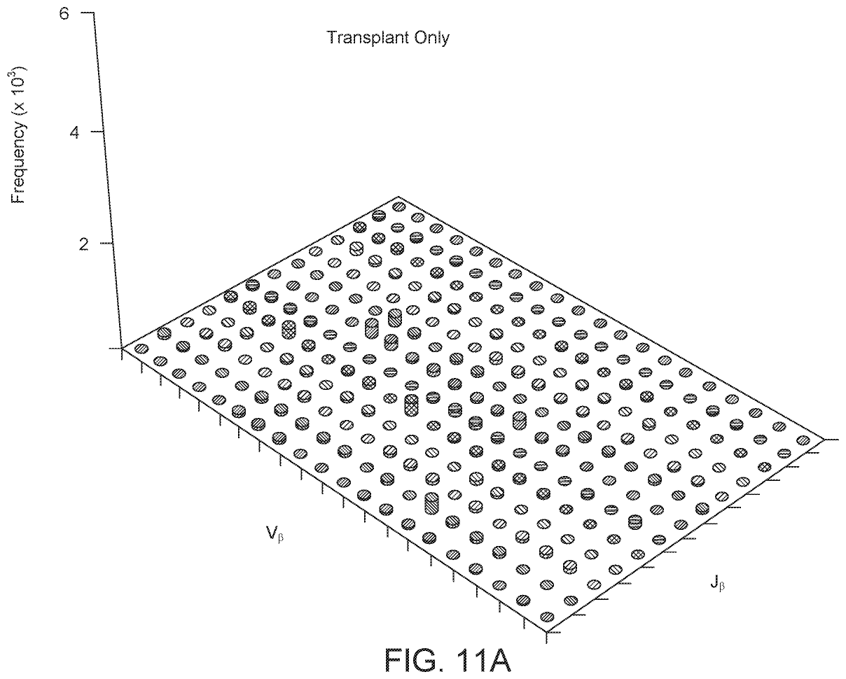

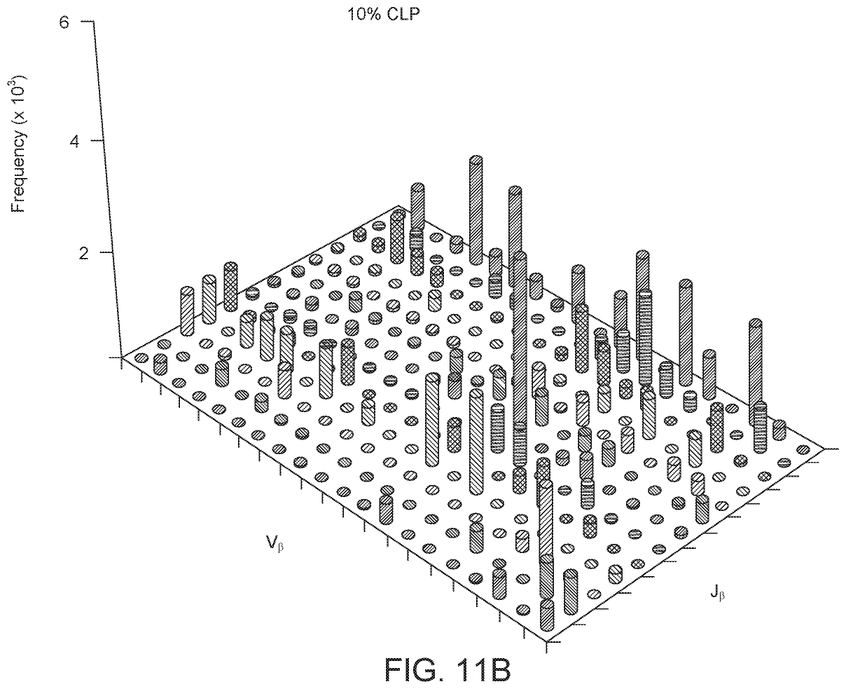

Practicing aspects of the system, device and method described herein, can generate bone marrow culture that are significantly enriched in CPLs. Shown in FIGS. 11A and 11B, are data for mice receiving bone marrow transplants supplemented with 10% CLPs (FIG. 11B). These mice exhibited greater diversity in their T cell repertoires than mice receiving untreated bone marrow (FIG. 11A). The plots show the distribution of T Cell Receptor V and J segments in the CDR3 β chain of transplanted mice using Simpson's index, which takes into account the total number T cells present as well as their relative abundance. The greater the number of bars, the higher the diversity; the taller the bars, the higher the number of clones.

While the method 600 is illustrated in FIGS. 7-9 as being performed with a microfluidic micro-channel cassette 102, the method 600 (or a similar method) can be performed with a well-based cassette 102.

For example, a first fluid, which can include a bone marrow, mobilized peripheral blood or cord blood suspension, that includes a population of HSCs can be pumped, flowed, or otherwise provided to the one or more wells of the cassette 102. The cell population can be distributed across the trapping surface 212 by the distribution system 300. The trapping surface 212, containing a plurality of notch ligands, can cause the HSCs to differentiate into CLPs over a period of about 3 to about 7 days. The CLP-enriched cell population can then be collected from the well-based cassette 102. For example, the distribution system 300 can induce a shear force in the fluid within the well 302 to dislodge the CLPs (and other cells) from the trapping surface 212. The CLP-enriched cell population can be collected for implantation into a patient.

Applications other than bone marrow transplantation exist as well. For example, techniques described herein could be used as an autologous approach for radiation protection. In one illustration, a subject, a soldier, for instance, could band his or her cells prior to deployment in a situation that may involve exposure to radiation. With the added CLPs, the subject's immune system could more quickly reconstitute with increased diversity. In many cases, the method, system and/or device are used in a hospital setting, by medical professionals.

While this invention has been particularly shown and described with references to preferred embodiments thereof, it will be understood by those skilled in the art that various changes in form and details may be made therein without departing from the scope of the invention encompassed by the appended claims.

What is claimed is:

1. A system comprising:
a device including a trapping surface configured to receive hematopoietic stem cells (HSCs), wherein the device includes at least one cell well or at least one microchannel, the at least one cell well or the at least one microchannel containing a trapping surface comprising a notch ligand for inducing differentiation of the HSCs into common lymphoid progenitors (CLPs), and
a processor for controlling flows to and from the device and/or conditions in the system.

2. The system of claim 1, further comprising one or more reservoirs, a microfluidic arrangement for supplying or removing fluids to and from the device, sensors for determining system conditions, optionally, a source for supplying acoustic radiation, or any combination thereof.

3. The device of claim 1, wherein the notch ligand is a notch ligand Delta-like 4 (DLL4).

4. The device of claim 1, wherein the notch ligand is attached to the trapping surface by physical adsorption, capture by immobilized anti-DLL4 antibody, or by covalent coupling.

5. The system of claim 1, wherein the device is a well-based cassette, optionally including a distribution system.

6. A microfluidic cassette comprising at least one cell well containing a trapping surface that includes a notch ligand, and a distribution system for providing cells or fluids to the at least one cell well, wherein the cassette or the distribution system is configured to rotate.

7. The microfluidic cassette of claim 6, wherein the distribution system is a perforated disk, an impeller or a centrifuge.

8. The microfluidic cassette of claim 6, wherein the distribution system comprises a disk having a first face and a second face, wherein the first face comprises an inlet and the second face comprises a plurality of outlets configured to enable passage of the population of hematopoietic stem cells, and wherein the disk is configured to rotate within a cell well.

9. The microfluidic cassette of claim 6, wherein the distribution system comprises at least one impeller configured to rotated within each of one or more cell wells to generate a shear force in a fluid in each of the one or more cell wells.

10. The microfluidic cassette of claim 6, wherein the notch ligand is a notch ligand Delta-like 4 (DLL4) that is attached to the trapping surface by physical adsorption, captured by immobilized anti-DLL4 antibody, or by covalent coupling.

11. A device comprising:
a first microfluidic channel;
a membrane between the first microfluidic channel and a second microfluidic channel,
wherein the membrane comprises pores smaller than a diameter of a hematopoietic stem cell,
wherein the second microfluidic channel comprises a trapping surface opposite the membrane, and
wherein the trapping surface comprises a notch ligand.

12. The device of claim 11, wherein the notch ligand is configured to induce differentiation of hematopoietic stem cells (HSCs) into common lymphoid progenitors (CLPs).

13. The device of claim 11, wherein the notch ligand is a notch ligand Delta-like 4 (DLL4) that is attached to the trapping surface by physical adsorption, captured by immobilized anti-DLL4 antibody, or by covalent coupling.

14. A method comprising:
flowing a first fluid comprising hematopoietic stem cells (HSCs) through a first microfluidic channel and into a second microfluidic channel via a membrane disposed between the first microfluidic channel and the second microfluidic channel, wherein the membrane has pores that are smaller than the HSCs;
capturing HSCs on a first face of the membrane;
distributing the HSCs onto a trapping surface opposite the membrane, wherein the trapping surface comprises a notch ligand configured to induce differentiation of the captured HSCs into common lymphoid progenitors (CLPs);
flowing a second fluid through the second microfluidic channel to provide nutrients and/or oxygen into the first microfluidic channel; and
flowing a third fluid through the first microfluidic channel to wash the CLPs from the trapping surface.

15. The method of claim 14, wherein the notch ligand is a notch ligand Delta-like 4 (DLL4) that is attached to the trapping surface by physical adsorption, captured by immobilized anti-DLL4 antibody, or by covalent coupling.

16. The method of claim 14, wherein the HSCs are distributed onto the trapping surface by gravity.

17. The method of claim 14, wherein the CPLs are detached from the trapping surface by a shear force in the third fluid.

18. The method of claim 14, wherein the method is controlled by a processor.

19. The method of claim 14, wherein the method is fully automated.

20. The method of claim 14, further comprising sensing flow rates, temperatures, and/or a culture medium composition.

21. The method of claim 14, wherein at least one of the first, second or third fluids is supplied from a reservoir.

22. The method of claim 14, wherein the first face of the membrane forms a surface of the first microfluidic channel and wherein a nutrient is perfused through the membrane.

23. A method comprising:
establishing an acoustic standing wave in a microfluidic channel;
allowing hematopoietic stem cells (HSCs) to distribute at nodes or antinodes of the standing wave;
trapping the distributed HSCs onto a trapping surface that includes a notch ligand configured to promote differentiation of HSCs to lymphoid progenitors (CLPs); and
separating CPLs from the trapping surface.

24. The method of claim 23, wherein the trapping surface is provided on micro- or nano-beads.

25. The method of claim 24, wherein the micro- or nano-beads beads have magnetic properties.

26. A method for treating a subject, the method comprising:
administering to a subject in need of a bone marrow transplant CPLs obtained by a method including:
flowing a first fluid comprising hematopoietic stem cells (HSCs) through a first microfluidic channel and into a second microfluidic channel via a membrane disposed between the first microfluidic channel and the second microfluidic channel, wherein the membrane has pores that are smaller than the HSCs;
capturing HSCs on a first face of the membrane;
distributing the HSCs onto a trapping surface opposite the membrane, wherein the trapping surface comprises a notch ligand configured to induce differentiation of the captured HSCs cells into CLPs;
flowing a second fluid through the second microfluidic channel to provide nutrients and/or oxygen to the first microfluidic channel; and
flowing a third fluid through the first microfluidic channel to wash the CLPs from the trapping surface.
